(19) United States
(12) Patent Application Publication
(10) Pub. No.: US 2024/0353038 A1
(43) Pub. Date: Oct. 24, 2024

(54) COUPLING ELEMENT FOR AN IMPROVED STERILE FLANGE CONNECTION

(71) Applicant: ZETA GmbH, Lieboch (AT)

(72) Inventors: Michael KOGLER, Stainz (AT); Georg PÖSCHL, Semriach (AT); Birgit PITTERMANN, Preding (AT); Reinhard WEINGARTSHOFER, Hitzendorf (AT)

(73) Assignee: ZETA GmbH, Lieboch (AT)

(21) Appl. No.: 18/637,808

(22) Filed: Apr. 17, 2024

(30) Foreign Application Priority Data
Apr. 18, 2023 (AT) A 50286/2023

(51) Int. Cl. F16L 23/16

(52) U.S. Cl. CPC F16L 23/16

(57) ABSTRACT

A coupling element for a sterile flange connection for connecting fluid channels, comprising a fluid-contacting surface, a first opening end and a second opening end of the fluid-contacting surface, a flange part at the first opening end and/or at the second opening end, a connection plane, the connection plane being arranged normal to an axial coupling element axis of the coupling element and occluding the respective opening end of the coupling element in a delimiting manner, and a film, the film closing at least the first opening end of the fluid-contacting surface, with the coupling element comprising at least one film holding element separate from the coupling element and comprising a film-contacting surface to which the film is removably connected.

### CROSS-REFERENCE TO RELATED APPLICATIONS

This U.S. application claims the benefit of Austrian Application No. A 50286/2023, filed Apr. 18, 2023. The entire content of the priority application is incorporated herein by reference.

The invention relates to a coupling element for a sterile flange connection for connecting fluid channels, comprising a fluid-contacting surface, a first opening end and a second opening end of the fluid-contacting surface, a flange part at the first opening end and/or at the second opening end, a connection plane, the connection plane being arranged normal to an axial coupling element axis of the coupling element and occluding the respective opening end of the coupling element in a delimiting manner, and a film, the film closing at least the first opening end of the fluid-contacting surface.

Products such as pharmaceutical active substances that are produced biotechnologically are subject to strict legal requirements which are supposed to ensure the quality of the products. Highly sterile conditions must thus be created during production, storage and transport. Such requirements for sterile conditions should also be met when guiding liquids between material containers, processing and analytical devices, whereby the risk of introducing unwanted contamination into products arises particularly when connecting or, respectively, coupling one fluid channel to another fluid channel. The establishment of a connection of fluid channels which is detachable and connectable multiple times, while preserving sterility, is therefore of particular importance, since an entry point for contamination is provided by the generally non-sterile environment, especially when a connection is created. Contamination can be understood to be unwanted contaminants, including biological contaminants such as viruses or tiny organisms, e.g., bacteria, and environmental contaminants such as dust or dirt. Such contamination can be extremely harmful for a variety of reasons.

After the connection of fluid channels and prior to guiding any product, a cleaning cycle is typically run through the product manufacturing facility, which may involve multiple cleaning agents. In doing so, acids and alkalis are conveyed consecutively through fluid channels, separately from rinses with water and steam, until sterility is achieved. This requires considerable time and energy, which consequently results in a less economical product manufacturing process and therefore constitutes a significant disadvantage. If the lines could be kept sterile even as they are being connected, fewer cleaning cycles would have to be run through the product manufacturing facility.

However, it is not only important to prevent contamination from entering into fluid channels and to reduce cleaning cycles. The simple and safe connection of fluid channels is also of particular importance, since material containers in particular are regularly connected to and disconnected from a production facility, involving significant economic consequences. It is therefore important that the coupling of fluid channels is as easy as possible for a user to perform in order to save time and also, above all, to be certain in the process that no contamination has been able to enter into the coupling while the user manipulates and connects the coupling element.

Sterile coupling elements according to the prior art generally comprise a removable film in the area of the opening ends. This film is stuck to the coupling elements by means of an adhesive, although this film can also be welded, pressed or shrunk on. A known system according to the prior art is disclosed in U.S. Pat. No. 8,454,059 B2.

The coupling element of the present document is an advanced development of the sterile coupling element disclosed in EP 3 992 516 A1. In this patent application, a coupling element is shown the opening end of which can be closed with a film.

A significant disadvantage of existing solutions from the prior art is that, when the coupling elements are being manipulated or, respectively, handled, there is a risk of the film becoming detached accidentally or prematurely, and thus of contamination being introduced into the elaborately sterilized fluid channel and connection area.

It is therefore the object of the invention to provide an improved coupling element for a sterile flange connection for connecting fluid channels, whereby the above-mentioned disadvantages of known coupling elements are to be eliminated. Primarily, the disadvantage of the film becoming detached in a premature, accidental manner is to be avoided.

According to the invention, the present object is achieved in that the coupling element comprises at least one film holding element separate from the coupling element and comprising a film-contacting surface to which the film is removably connected.

As a result of the at least one film holding element, the advantage is obtained that the film is additionally or exclusively connectable to the separate film holding element, with a more reliable connection of the film to the coupling element being achieved by the film holding element. In this way, it is effectively achieved that the film will not be accidentally detached by a user manipulating the coupling element, because the adhesion of the film to the coupling element is not sufficient. As a result, a significantly improved protection against ingress of contamination is obtained. A partial detachment of the film is particularly critical, as this can easily be missed by the user since the film gives the impression that the film is still completely connected to the film holding element.

Firstly, the term flange part can be understood as denoting a part connected to the coupling element. In this case, the flange part can exist in a glued or welded state. Secondly, the monolithic embodiment is also included therein. The flange part can also exist as a loose flange part, in which case a thickening is required at the respective opening end so as to enable a connection.

Preferably, at least one sealing element surrounding the first opening end is formed in the area of the connection plane. As a result, the advantage is obtained that, during the assembly to form a flange connection, an additional barrier is provided against ingress of contamination into the connection of the fluid channels.

The area of the connection plane is understood to mean that the thickness of the sealing element intersects, or at least touches, the connection plane. This is the only way of providing a tight and sterile connection when the coupling element is coupled. When the film is removed during the process of coupling the coupling element, a gap might furthermore arise, this gap being closed immediately by the sealing element in order to thus preserve continuous sterility during the connection of fluid channels. In general, the coupling element is lightly pressed against another connection plane during coupling so that the film can still be removed by a user without creating an entry point for contamination.

The at least one sealing element is preferably formed in at least one sealing element recess, the sealing element extending beyond the connection plane and the sealing element recess being incorporated into the flange part. The advantage associated therewith is that the sealing element is held in the coupling element by a positive fit, without the need to provide any other type of attachment. The sealing element recess can preferably coincide with the shape of the sealing element. In this case, a milled sealing element recess with a circular cross-section can be provided for an annular O-ring sealing element, although other shapes of the sealing element recesses may be provided, too. Another type of sealing element may also be present, such as profile seals or sealants, for example.

Preferably, the at least one film holding element is also formed in the area of the connection plane. As a result, the advantage is obtained that the film can be designed with a small surface area, whereby the occurrence of possible defects in the film, through which contamination could enter, can be reduced. For example, if the film holding element is not provided in the area of the connection plane, a major part of the coupling element is indeed sterile-packed, but a major part of the film is also exposed to possible damage or defects which may occur in a production process of the film.

According to one embodiment, the at least one film holding element is formed in at least one film holding element recess, the film-contacting surface of the film holding element ending with the connection plane, with the film holding element recess being incorporated into the flange part. By ending with the connection plane, there are no edges on which the film could get damaged when the coupling element is being manipulated, since the film lies flat on the connection plane without any edges being formed. In addition, the advantage is thus obtained that, when the coupling element is coupled, no edge or, respectively, corner of the film holding element recess can enter into a selective contact, whereby the robustness of the coupling element can be improved, since the film holding element recess cannot be knocked out. In the event of a knockout, material the coupling element is made of could be introduced into a product, which, in turn, constitutes critical contamination.

According to a further embodiment wherein a sealing element and a film holding element are formed in the area of the connection plane, a circumference of the film holding element is larger than a circumference of the sealing element. As a result, the advantage is obtained that the sealing element is covered with the film, whereby a particularly sterile coupling element is obtained, since fluid is retained by the sealing element in the operating state and thus cannot come into contact with the flange part outside of the sealing elements, whereby the surface area of possible contamination is reduced. In addition, the sealing element first comes into contact with another connection surface, when the coupling element is coupled. During coupling, the sealing element thereby presses the film against another connection plane, whereby the connection already has a certain tightness and thus protection against ingress of contamination when the film is being removed.

Preferably, the film holding element is made of plastic, preferably polyethylene (PE), and/or the coupling element is made of metal, preferably steel, especially polished steel. Films, especially those designed from a composite material, adhere particularly well to the material polyethylene, with polyethylene being particularly well suited for sterile coupling elements due to its properties, as this material exhibits high tenacity, wet and tear strength, multiple usability and easy processability. A particularly good connection between the film holding element and the film is important especially if the coupling element is made of metal, since the film generally has a particularly poor adherence to metal. This fact is enhanced if the coupling element is made of polished steel, since, in this case, there is almost no adhesion of the film to the metal surface. Coupling elements made of polished steel are particularly preferred because they are often reusable and because they exhibit great mechanical strength compared to synthetic materials and other materials. Furthermore, the polished, smooth surface causes further worsening of the accumulation of contamination on the surface and the fluid-contacting surface. Hence, the film holding element according to the invention is particularly advantageous for the coupling element made of polished steel.

It should also be mentioned that the film holding element can be created from pharma-compliant and other synthetic materials. Of those, the following synthetic materials should be mentioned, for example: PE 1000 PURE, PPSU, PES, PSU, PVDF, PET, POM-H, POM-C, PA 6 G 210, PA 66, PA 6, PP, PE-UHMW, PE 500, PE 300 and PE 100.

According to one embodiment, at least one through hole is formed in the flange part, the film holding element being accessible via the through hole. Due to this through hole, the advantage is obtained that the film holding element can be pushed out of the coupling element. If there are signs of wear, a worn-out film holding element can thus be replaced by a new one particularly easily. Simply removing the film holding element is advantageous also if the coupling element is to be sterilized particularly thoroughly. Since it is difficult for a sterilizing agent to penetrate into a gap area between the inserted film holding element and the coupling element. However, if the film holding element can simply be removed from the coupling element, both components can be reliably cleaned without formation of a gap area.

The through hole can provide a significant advantage also when forming the film holding element, since, when the film holding element is injected via the through hole, there is an escape path for air, whereby inclusion of air can be effectively prevented. Air entrapment can cause indentations in the surface of the film holding element, with a film being unable to adhere thereto. The mechanical strength of the film holding element is also impaired by inclusions, which can lead to failure during operation.

Injection can also happen directly via the through hole. If the coupling element thereby rests with its connection plane against a working surface, the film holding element can thus be formed by direct injection into the through hole without an additional tool mould.

According to a further embodiment, the film holding element recess forms barbs. The barbs facilitate the grip of the film holding element in its film holding element recess, since the barbs provide an effective positive connection to the coupling element.

The film holding element preferably has at least one circumferential groove, wherein the groove can preferably be designed with a V-shape in the film-contacting surface and/or in a surface adjacent to the film-contacting surface and/or in a surface facing away from the film-contacting surface.

In any case, the circumferential groove has the advantage that stresses caused by temperature changes can be reduced by the groove. Furthermore, easier installation of the film holding element in the film holding element recess is achieved, since the circumferential groove provides greater formability.

If the groove is formed in the film-contacting surface, it is possible to effectively determine by way of the size of the groove as to how much force a user has to apply in order to pull off the film from the film-contacting surface. On the one hand, the film holding element must have a certain size so that it can be retained in the film holding element recess without breaking when the film is removed or without being pulled off together with the film. On the other hand, the surface to which the film is connected must not be too large so that it will be non-detachable or, respectively, unremovable as its contact area is too large. A groove in the film-contacting surface takes both criteria into account and can be dimensioned accordingly.

If the groove has a V-shaped design, another advantage obtained is that a knife for separating the film can be guided in this groove by a user without running the risk of scratching the connection plane of the coupling element. This is advantageous especially if the film is not provided in a suitable size for closing the opening ends and has to be cut to size on the coupling element itself. This is advantageous because contamination can be introduced into the unconnected part of the film, if the film protrudes beyond the film holding element when manipulating the coupling element. When the film is removed, contamination could be introduced by stripping these contaminated unconnected parts or, respectively, the non-sterile sides across the sealing elements or by touching the fluid-contacting surface, which consequently results in a less sterile connection.

It is another advantage that, if the film holding elements are not injected directly into the film holding recesses, but are manufactured externally, they can be deformed more easily due to the V-shaped groove in order to thus be pressed into the film holding recess. The side walls appropriately act like springs which retain the holding element by pressing it against the side wall of the film holding recess.

According to one embodiment, a frustoconical widening is formed in the area of the first opening end. This involves the advantage that material is thus provided which compensates for the loss of mechanical strength, which is obtained by providing the film holding element. The frustoconical widening is particularly advantageous if the sealing element recesses and the film holding recesses are designed, since the latter entail a significant reduction in the strength of the coupling element. Due to the weight of hose lines, large moments act on coupled coupling elements, for which reason it is essential to provide mechanical strength. Another advantage of the frustoconical widening is that the frustoconical shape can follow the bracing angle of the film, if the film is connected to the film holding element and if the film holding element is not arranged in the area of the connection plane. As a result, there are no areas on the coupling element that are spanned by the film. The film is thereby better protected against external influences, as the frustoconical widening is located directly behind the film.

Preferably, two coupling elements according to the invention and one connecting element for releasably connecting the two coupling elements can be provided in a flange connection. As a result, the advantage is obtained that a particularly sterile connection of fluid channels can be provided, since the interaction of two coupling elements according to the invention with their film holding elements highly effectively prevents the introduction of contamination due to the improved adhesion of the films to the film holding elements.

The coupling element according to the invention can be coupled to form a sterile flange connection as described below. In doing so, a film is connected at first to the film holding element. Next, the film is sealed under pressure and at a high temperature. Afterwards, the fluid-contacting surface of the coupling element is sterilized with steam. Subsequently, the first opening end is arranged essentially congruent with the opening end of a further coupling element. Afterwards, the coupling elements are accommodated at least in sections in the area of the opening ends by a connecting element. Subsequently, the films that close the opening ends and can be removed from the respective opening end are passed through a cavity in the connecting element. The sterile fluid connection is then established by simultaneously pulling off the films through the cavity of the connecting element. In the last-mentioned step, the films are pulled off from the opening ends through the cavity in the connecting element in order to produce the sterile fluid connection. Due to the connecting element, the advantage is obtained that the coupling elements are accommodated before the films are pulled off through the cavity in the connecting element. In this way, it is ensured that a secure, sterile and mechanically stable connection of the fluid channels exists before the films are removed. This is achieved in particular also by the fact that, in this case, the non-sterile sides of the films which abut against the opening ends do not touch the sterile or, respectively, low-germ fluid-contacting surfaces. As a result, foreign matter and/or germs are effectively prevented from entering into the fluid connection in the course of the removal of the films, thereby ensuring the sterility of the fluid connection. In addition, the fluid connection is mechanically stabilized even before the films are pulled off and is also secured against large mechanical loads, whereby the safety of the fluid connection is additionally increased. By designing the connecting element separately from the coupling elements, the fluid connection can again be separated simply by removing the connecting element. Upon cleaning, sterilization and the attachment of new films to the film holding element, the system is ready for re-use. If the film holding elements wear out after repeated use, they can be replaced easily without having to interfere with the substance of the coupling elements themselves. In this way, economic efficiency is increased and environmental compatibility is improved, whereby a sequence of coupling elements that is always securely connected is provided.

Advantageous and non-limiting embodiments of the invention are explained in further detail below with reference to the figures.

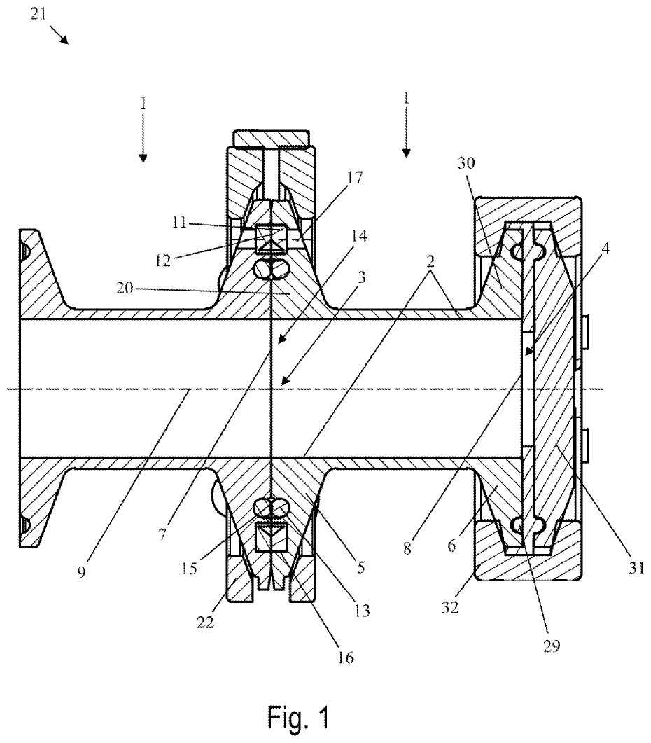

FIG. 1 shows a flange connection comprising two coupling elements that are coupled on their respective first connection planes, with a cover being coupled on a second connection plane of the right-hand coupling element via a further connecting means.

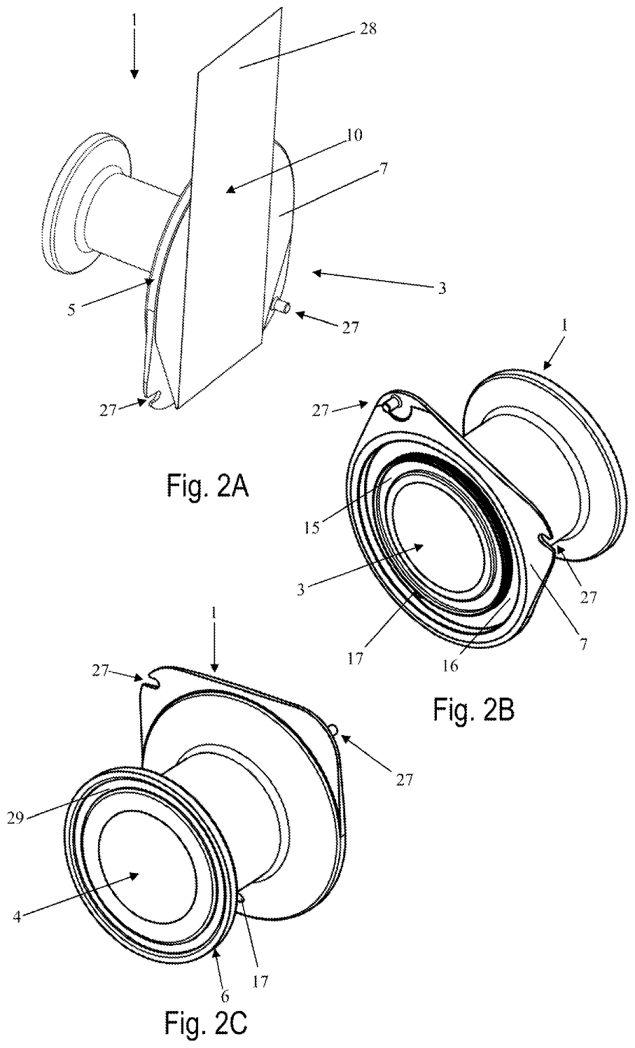

FIG. 2A shows the coupling element or, respectively, the other coupling element of FIG. 1 detached from the flange connection, isolated, in a first isometric view, with a film, the film closing the first opening end of the fluid-contacting surface.

FIG. 2B shows the coupling element of FIG. 2A without a film, in a second isometric view, wherein a first opening end and its connection plane are visible.

FIG. 2C shows the coupling element of FIG. 2B, in a third isometric view, wherein the second opening end is visible.

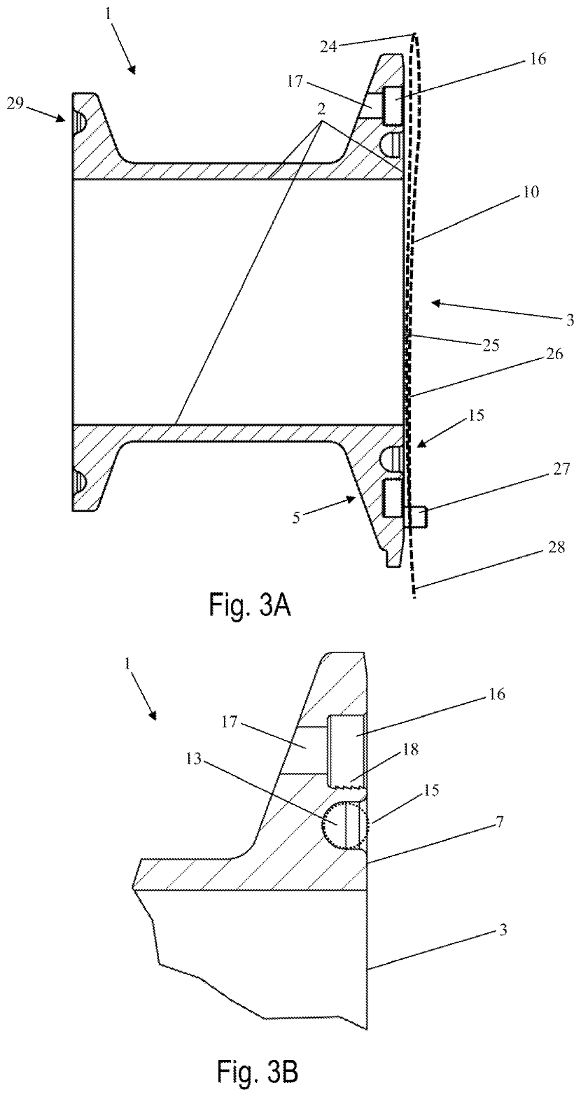

FIG. 3A shows the coupling element of FIGS. 2A to 2C in a sectional view.

FIG. 3B shows the sectional view of the coupling element of FIG. 2A, wherein the film holding element recess and the sealing element recess are shown in detail as an enlarged section.

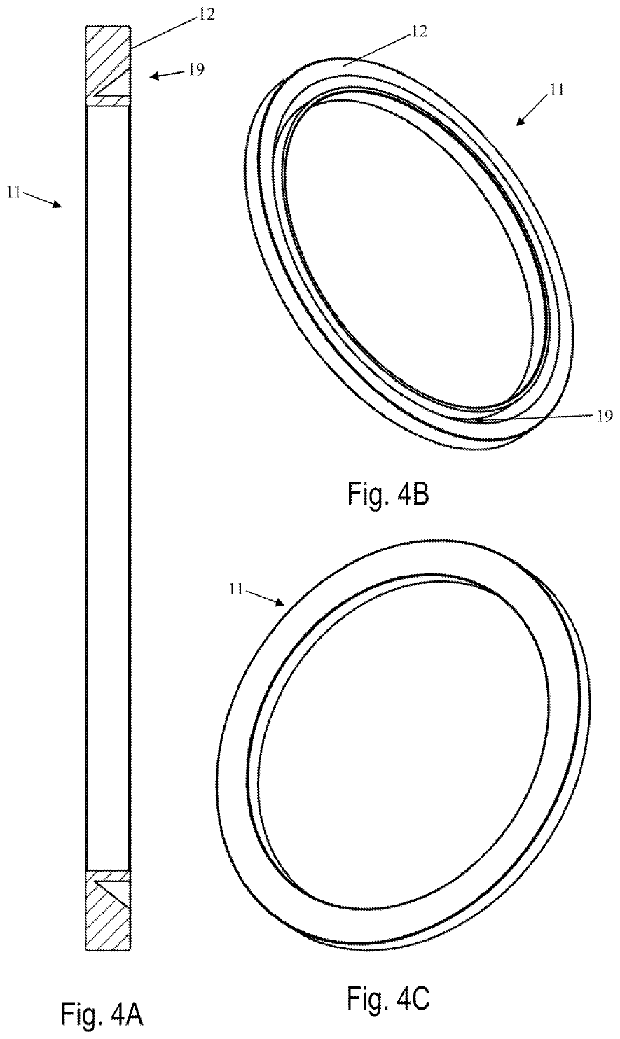

FIG. 4A shows the film holding element of FIG. 1 as being isolated in a sectional view.

FIG. 4B shows the film holding element of FIG. 4A in a first isometric view.

FIG. 4C shows the film holding element of FIG. 4A in a second isometric view.

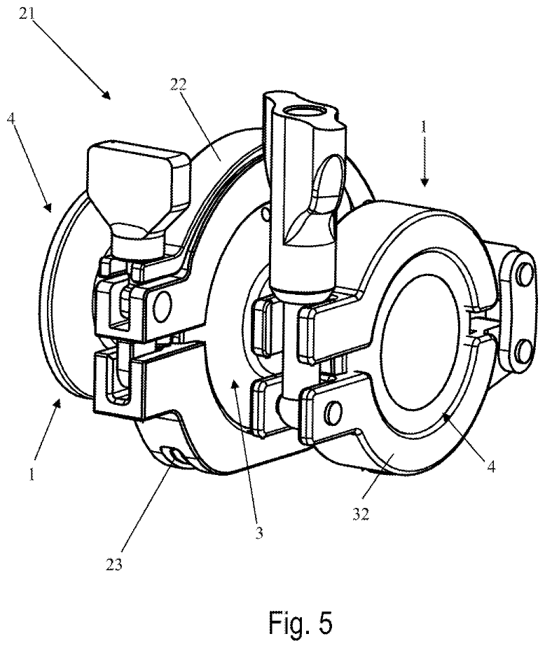

FIG. 5 shows the flange connection of FIG. 1 as being uncut in an isometric view.

FIG. 1 shows a detachable flange connection comprising a first coupling element 1 according to the invention and a further coupling element 1 according to the invention.

In this connection, it should be noted that the following explanations refer to that one of the two coupling elements which is on the right, although it will be obvious to a person skilled in the art that the explanations are applicable to the other coupling element on the left. Both coupling elements share reference numeral 1, as they are constructed identically in the flange connection 21 as illustrated in FIG. 1 and FIG. 5. In this case, it is obvious to a person skilled in the art that the other coupling element does not necessarily have to be designed in a structurally identical manner.

The two coupling elements 1 are brought into contact and coupled at their respective first connection planes 7, the releasable coupling being provided via a connecting element 22. A cover 31 is detachably coupled to the right-hand coupling element 1 on a second connection plane 8 via a further connecting element 32.

The coupling element 1 for the sterile flange connection 21 shown in FIG. 1 for connecting fluid channels comprises a fluid-contacting surface 2, a first opening end 3 and a second opening end 4 of the fluid-contacting surface 2, a flange part 5 at the first opening end 3 and a flange part 6 at the second opening end 4. The connection plane 7 is normal to an axial coupling element axis 9 of the coupling element 1, and the first opening end 3 occludes the coupling element 1 in a delimiting manner. In this embodiment, occluding in a delimiting manner means that no part of the coupling element 1 protrudes beyond the connection plane 7.

The coupling element 1 has at least one film holding element 11 separate from the coupling element 1 and comprising a film-contacting surface 12 to which a film 10 is removably connected. The film 10 is not illustrated in FIG. 1, since the coupling state is shown after the films 10 have been removed and the connecting element 22 has been closed. In other words, the two coupling elements 1 already communicate with each other.

In the coupling element 1, a sealing element 13 surrounding the first opening end 3 is formed in the area 14 of the connection plane 7. The sealing element 13 is formed in a sealing element recess 15, the sealing element 13 extending beyond the connection plane 7 and the sealing element recess 15 being incorporated into the flange part 5. Furthermore, the coupling element 1 has a second sealing element recess 29 in a flange part 6 at the second opening end 4, with no sealing element being shown therein, since the flange connection 21 is illustrated in the general context of product manufacture as being detached from its actual operating position. In general, the first coupling element 1 is coupled to a container with a product component via the flange part 6 at the second opening end 4. The other coupling element 1 is generally coupled to the rest of the product manufacturing facility via a flexible hose line.

The film holding element 11, like the sealing element 13, is formed in the area 14 of the first connection plane 7, the film holding element 11 being provided in a film holding element recess 16, the film-contacting surface 12 of the film holding element 11 ending with the first connection plane 7. The film holding element recess 16 is incorporated into the flange part 5.

A circumference of the film holding element 11 is larger than a circumference of the sealing element 13. In the context of a sealing element 13, a circumference can be understood to be its inner diameter, wherein the term can be expanded to include non-round sealing elements.

The sealing element 13 can, for example, also be designed as a flat seal, with the surface boundary thereof being understood to be the circumference.

The coupling element 1 has a through hole 17 which is formed in the flange part 5, with the film holding element 11 being accessible via the through hole 17. Accessible can be understood to mean that a user can touch the film holding element 11 with a tool through the through hole 17.

In the area 14 of the first opening end 3, a frustoconical widening 20 is formed, with, in this embodiment, a second frustoconical widening 30 also being formed in the area of the second opening end 4.

The flange connection 21 is preferably designed for a pressure range of 0 to 10 bar pressure in the fluid connection. According to the embodiment according to the invention, the coupling element 1 and the other coupling element 1 can also be connected to a hose and can be disinfected during use.

FIG. 2A shows the coupling element 1 or, respectively, the other coupling element 1 of FIG. 1 in a state of being detached from the flange connection 21, isolated, in a first isometric view, with a film 10, with the film 10 being depicted in contrast to FIG. 1. The film 10 closes the first opening end 3 of the fluid-contacting surface 2. The film 10 is connected to a first connection plane 7, which is arranged normal to an axial coupling element axis 9 of the coupling element 1. A graspable surface 28 of the film 10 thus protrudes beyond the flange part 5 so that a user can grasp this graspable surface 28 of the film 10 and pull it off.

The film 10 is preferably designed as a composite film made of commercially available, pharma-grade and/or food-safe materials, which is prefabricated in a punching machine. In this way, the coupling element 1 can be used in a wide pressure and temperature range. The prefabricated film 10 is preferably applied to the film holding element 11 at the opening end 3 with the aid of pressure and/or temperature. Due to the heat, the material of the film 10 melts and can be removed after cooling without leaving any residues.

FIG. 2B shows the coupling element 1 of FIG. 2A without a film 10, or, respectively, the one of FIG. 1, in a second isometric view, wherein the first opening end 3 and its connection plane 7 are visible. The film holding element recess 16 is depicted without a film holding element 11. The sealing element recess 15 is also depicted without a sealing element 13.

FIG. 2C shows the coupling element 1 of FIG. 2B, in a third isometric view, wherein the second opening end 4 is visible. The second sealing element recess 29 in the second flange part 6 at the second opening end 4 is again depicted without a sealing element.

FIG. 3A shows one of the two coupling elements 1 of FIG. 1 to FIG. 2C in a sectional view. No sealing elements 13 and no film holding element 11 are depicted therein in the sealing element recesses 15, 29 and in the film holding element recess 16. However, a film 10, which closes the first opening end 3, is illustrated by dashed lines. In this case, the film 10 is folded once along its broad side so that a graspable surface 28 protrudes downwards beyond the flange part 5. As a result, the advantage is obtained that, when coupling in a flange connection 21 (as illustrated in FIG. 1), the two films 10 in contact can be unrolled from each other, and the film 10 can be detached particularly easily by shearing in the deflection area 24 or fold line, respectively, when unrolling from the film holding element 11. Furthermore, possible contamination that has accumulated on a non-sterile side 26 of the film 10 is thus reliably carried away from the fluid-contacting surface 2, without the risk of the non-sterile side 26 of the film 10 touching the sterilized fluid-contacting surface 2 and introducing contamination. It should be noted that the non-sterile side 26 of the film 10 is understood to be the entire part that is exposed to the environment. Accordingly, the sterile side 25 of the film 10 is the part of the film 10 which communicates with the fluid-contacting surface 2 and is provided in a particularly sterile manner in the course of a sterilization of the coupling element 1.

FIG. 3B shows the coupling element 1 of FIG. 2A in a sectional view, wherein the film holding element recess 16 and the sealing element recess 15 are shown in detail as an enlarged section. In FIG. 3B, it can be seen particularly clearly that the film holding element recesses 16 form barbs 18. Furthermore, the sealing element recess 15 is also easier to identify. In the sealing element recess 15, a sealing element 13 of the O-ring type is schematically illustrated with a dotted line, which is supposed to highlight that the sealing element 13 extends minimally beyond the connection plane 7. It should be mentioned that the barbs 18 can be designed in a variety of ways. For example, only a single tooth-shaped projection can be provided for keeping the film holding element 12 in the film holding element recess 16. However, several projections can also be designed so as to act as barbs 18. It should also be mentioned that the barbs 18 can be provided on any surface of the film holding element recess 16. Likewise, the barbs 18 do not have to run around the entire film holding element recess 16, but can only be provided at certain points. For example, at three points in the film holding element recess 16, wherein these points can be spaced apart from each other by 120°.

FIG. 4A shows the film holding element 11 of FIG. 1 as being isolated in a sectional view. The film holding element 11 preferably has a circumferential groove 19, the groove 19 preferably being designed with a V-shape in a film-contacting surface 12. The film holding element 11 is a part separate from the coupling element 1. Therefore, the film holding element 11 can be made of a different material than the coupling element 1, whereby the advantage is obtained that the film holding element 11 can be created from a material which adheres particularly well to a film 10, without the need to create the entire coupling element 1 from such a material. In this case, the film holding element 11 can preferably be comprised of a synthetic material. However, the film holding element 11 can also be created from the same material as the coupling element 1, wherein the film-contacting surface 12 can provide a different surface. For example, the film-contact surface 12 can be roughened so that the film 10 can adhere to it better than to a smooth, polished coupling element 1, for example.

It should be particularly emphasized that the film holding element 11, in contrast to the orientation of the film holding element 11 of FIG. 1, can also be designed inversely in the film element recess 16 so that the film 10 lies flatly on the first connection plane 7, with the circumferential groove 19 being designed in a surface facing away from the film-contacting surface 12. In this connection, it should be mentioned that the coupling elements 1 of FIG. 1 can be designed in such a way that the left-hand coupling element 1 can have a film holding element 16 that has been inserted inversely, in contrast to what is illustrated in FIG. 1. In this case, the right-hand coupling element 1 can have a film holding element 11 which is oriented as shown in FIG. 1. In other words, the film holding element 11 can be placed in the film element recess 16 in any orientation.

FIG. 4B shows the film holding element 11 of FIG. 4A in a first isometric view so that its groove 19 can be seen. FIG. 4C, on the other hand, shows the film holding element 11 of FIG. 4A in a second isometric view so that its flat backside can be seen.

FIG. 5 shows the flange connection 21 of FIG. 1 in an isometric view. Therein, it is emphasized that the connecting element 22 and the further connecting element 32 can preferably be designed as a union clamp. This configuration of the connecting element 22 provides the advantage that a high biasing force can be provided when the first coupling element 1 and the other coupling element 1 are accommodated in the area of the opening ends 3, 4. As a result, the mechanical stability of the flange connection 21 is increased, and the risk of contamination when removing the films 10 is reduced. Furthermore, the design as a union clamp enables the connecting element 22 to be easily attached and detached and ensures a high contact pressure when coupling.

The separate connecting element 22 is designed for accommodating the coupling element 1 and the other coupling element 1 in sections in the area of the frustoconical widening 20. As part of the accommodation of the coupling elements 1 by the connecting element 22, the opening ends 3 closed with the films 10 (films 10 not shown) are pressed together. The connecting element 22 has a cavity 23 arranged essentially in the connection plane 7 for passing the films through the connecting element 22. Preferably, the cavity 23 has a slot-shaped design. The cavity 23 allows always the same peel angle of the films 10 to be maintained by a user. The connecting element 22, 32 is preferably made of steel.

It should be noted that the coupling elements 1 preferably have localization elements 27 that can be coupled to each other, which can be seen in FIG. 2A to FIG. 3B. The localization elements 27 are designed for positioning the first opening ends 3 essentially congruent with each other during the act of coupling. As a result, optimal positioning of the coupling element 1 and the other coupling element 1 is ensured. The localization elements 27 are preferably designed as grooves and pins, which always engage with each other.

It should be noted that, although in all figures only cylindrical coupling elements 1 with a circular cross-section are depicted, other cross-sectional shapes can be provided, too. For example, a rectangular cross-section should be mentioned.

Furthermore, it should be noted that coupling elements 1 of different embodiments can also be present in a flange connection 21. For example, only one of the two coupling elements 1 can have a sealing element 13, or the film holding elements 11 and/or the sealing elements 13 can be designed at different heights.

It should also be noted that the coupling element 1 can also have additional through holes with which a connection can be established. This includes, for example, bores arranged in the flange part through which screws, as connecting elements 22, can be passed in order to be connected to another coupling element 1.

Furthermore, it should be noted that several separate or, respectively, adjacent sealing elements 13 and/or film holding elements 11 can also be present in a coupling element 1. For example, one film holding element 11 cannot be located in the area of a connection plane 14 and another one can be located in the area of the connection plane 14. Both film holding elements 1 can be connected to a single film 10, or a second film 10 can be connected to the film holding element 1 not located in the area of the connection plane 13. In both cases, a particularly sterile coupling element 1 is obtained since a larger surface area is covered by a film 10. In other words, the film 10 or, respectively, the second film can be regarded, in a second function, as a kind of packaging for the coupling element 1, the packaging being connected to the coupling element 1.

Finally, it should be noted that it is obvious to a person skilled in the art that the film holding element 1 according to the invention can also be provided at the second opening end 4, just like the film holding element 1 of the first opening end 3. In this way, the advantage is obtained that both opening ends 3, 4 can be coupled in a sterile manner.

For example, the fluid-carrying surface 2 can be elongated more than what is shown in the figures. The same applies, for example, to the frustoconical widenings 20, 30, which can be provided at a different angle than what is shown.

1. A coupling element for a sterile flange connection for connecting fluid channels, comprising:
a fluid-contacting surface,
a first opening end and a second opening end of the fluid-contacting surface,
a flange part at the first opening end and/or at the second opening end,
a connection plane, the connection plane being arranged normal to an axial coupling element axis of the coupling element and occluding the respective opening end of the coupling element in a delimiting manner,
and a film, the film closing at least the first opening end of the fluid-contacting surface,
wherein the coupling element comprises at least one film holding element separate from the coupling element and comprising a film-contacting surface to which the film is removably connected.

2. The coupling element according to claim 1, wherein at least one sealing element surrounding the first opening end is formed in the area of the connection plane.

3. The coupling element according to claim 2, wherein the at least one sealing element is formed in at least one sealing element recess, the sealing element extending beyond the connection plane and the sealing element recess being incorporated into the flange part.

4. The coupling element according to claim 1, wherein the at least one film holding element is formed in the area of the connection plane.

5. The coupling element according to claim 4, wherein the at least one film holding element is formed in at least one film holding element recess, the film-contacting surface of the film holding element ending with the connection plane, with the film holding element recess being incorporated into the flange part.

6. The coupling element according to claim 2, wherein a circumference of the film holding element is larger than a circumference of the sealing element.

7. The coupling element according to claim 1, wherein the film holding element is made of plastic, preferably polyethylene, PE 1000 PURE, PPSU, PES, PSU, PVDF, PET, POM-H, POM-C, PA 6 G 210, PA 66, PA 6, PP, PE-UHMW, PE 500, PE 300 and PE 100, and/or that the coupling element is made of metal, preferably steel, especially polished steel.

8. The coupling element according to claim 5, wherein at least one through hole is formed in the flange part, the film holding element being accessible via the through hole.

9. The coupling element according to claim 5, wherein the film holding element recess forms barbs.

10. The coupling element according to claim 1, wherein the film holding element has at least one circumferential groove, wherein the groove is preferably designed with a V-shape in the film-contacting surface and/or in a surface adjacent to the film-contacting surface and/or in a surface facing away from the film-contacting surface.

11. The coupling element according to claim 1, wherein a frustoconical widening is formed in the area of the first opening end.

12. A flange connection comprising two coupling elements according to claim 1 and one connecting element for detachably connecting the two coupling elements.
